(19) United States
(12) Patent Application Publication
(10) Pub. No.: US 2012/0237967 A1
(43) Pub. Date: Sep. 20, 2012

(54) Method for Microbiological Testing

(76) Inventor: Mansour Samadpour, Lake Forest Park, WA (US)

(21) Appl. No.: 13/176,424

(22) Filed: Jul. 5, 2011

Related U.S. Application Data
(63) Continuation of application No. 12/803,820, filed on Jul. 6, 2010, now abandoned.

(51) Int. Cl. C12Q 1/04

(52) U.S. Cl. 435/34

(57) ABSTRACT

Costly enrichment medium used for microbiological testing of nuts and other moisture-absorbing food products is conserved by saturating the product with water or other inexpensive liquid prior to contacting the product with enrichment medium.

This application is based upon Provisional Application 61/269,469 filed Jul. 21, 2009 and is a continuation of Utility application Ser. No. 12/803,820 filed Jul. 6, 2010.

### BACKGROUND OF THE INVENTION

1. Field of the Invention

The present invention relates to methods for microbiological testing of powdered, granular, fibrous, or other products which have the capacity to absorb moisture, including food products such as almonds and pistachios. In particular, it relates to methods for economizing use of enrichment medium used during test procedures.

2. Description of the Related Art

Almonds and pistachios often need to be tested for harmful bacteria. To make tests for bacteria more effective, samples of nuts or nut kernels are soaked in a water solution containing nutrients, also known as an enrichment medium. The enrichment medium is typically designed to promote the growth of a particular bacteria, and it sometimes also discourages growth of competing bacteria. Due to specialized nutrient content and preparation steps required, enrichment media can be expensive. This is particularly so with products such as almonds and pistachios when testing protocols require large sample sizes. Testing with large samples requires a substantial volume of enrichment medium.

In prior art testing protocols, much enrichment medium is not efficiently used. Nuts tend to have interiors which are dry and capable of absorbing large amounts of liquid. Thus, when enrichment medium is added to a batch of nuts, much of it is absorbed into the interiors of the kernels. Since harmful bacteria are typically hosted on the exposed portions of nuts, not inside the solid kernels, prior art application of enrichment medium directly to nuts does not contribute to the testing process, with much of the medium being absorbed into nut interiors and therefore essentially wasted. The present inventor has observed that two volumes of enrichment medium added to one volume of nuts will sometimes result in only one-half volume of free liquid. Thus, 75% of the added medium ends up merely adding moisture to the interiors of the nut kernels, rather than providing a useful growth environment for the bacteria located upon the surfaces of the nut.

Thus, it is desirable that a way be found to conserve the use of expensive enrichment medium when doing laboratory testing of moisture-absorbing granular products, including food products such as nuts.

It is also desirable that a way be found to locate the expensive enrichment medium at the surfaces of the nut shells and kernels where it can do the most good, rather than inside the nut kernels where bacteria do not tend to exist anyway.

It is particularly desirable that a way be found to reduce the cost of laboratory testing of granular products by providing for a more efficient way to use expensive enrichment medium.

### SUMMARY OF THE INVENTION

It is, therefore, an object of the present invention to conserve the use of expensive enrichment medium when doing laboratory tests of moisture-absorbing food products such as nuts.

It is also an object of the present invention to locate the expensive enrichment medium at the surfaces of nut kernels and shells where it can do the most good, rather than inside the nut kernels where bacteria do not tend to exist anyway.

Further, it is an object of the present invention to minimize the cost of laboratory testing of granular products by providing for a more efficient way to use expensive enrichment medium materials. Lower cost tests will allow the industry to conduct more tests within the current food safety budget, thus improving overall food safety.

To achieve these and other objects, the present invention provides for pre-soaking, wherein nuts are pre-soaked with an inexpensive liquid such as water, so that the nut kernels become saturated. Then, when enrichment medium is added, the pre-soaked nut kernels are unable to absorb more liquid into their interiors, and the enrichment medium tends to bathe just the exterior of the nuts and nut kernels, rather than being absorbed into the interiors. Since the enrichment medium is now not being absorbed into the interiors of the nuts, much less medium is required to provide a liquid environment for that place on the nuts where bacteria are likely to be living, that is, on the surfaces of the nut shells and nut kernels. The inexpensive pre-soak liquid could be just pure water, however, it is preferable that a solute be added that will protect bacteria against osmotic shock and other harms. One preferable pre-soak liquid would be a 0.5% by weight solution of sodium chloride in water.

### BRIEF DESCRIPTION OF THE DRAWINGS

FIG. 1 is a diagram showing a sample of pistachios with a beaker of pre-soak liquid being added.

FIG. 2 is a diagram showing the same sample after the pre-soak liquid has been absorbed into the nuts, with the liquid level shown toward the bottom of the beaker.

FIG. 3 is a diagram showing the same sample after addition of enrichment medium, ready for incubation.

### DETAILED DESCRIPTION OF THE PREFERRED EMBODIMENTS

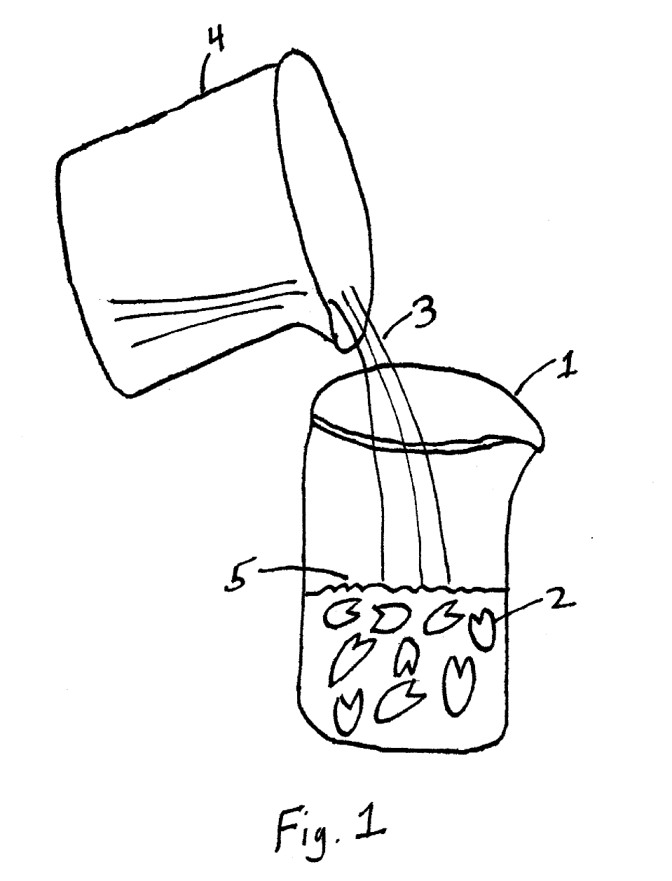

FIG. 1 is a diagram showing a sample of pistachios with a beaker of pre-soak liquid being added.

In FIG. 1, beaker 1 containing pistachio nuts 2 is shown being filled with pre-soak liquid 3 flowing from vial 4. The beaker is filled such that all the nuts are covered with liquid up to liquid level line 5. Pre-soak liquid 3 might comprise, for example, 99.5 parts of water, with 0.5 parts by weight of sodium chloride to provide an osmotic environment conducive to the survival of typical bacteria, such as Salmonella or E. coli. The amount of liquid added to the container will depend upon how dry the nuts are to begin with, where dry nuts are able to absorb more liquid and moist nuts less. For typical California pistachios, it has been found that at least a volume of liquid equal to the original volume of the nuts can be added. Absorbing water into the nuts is a slow process that may take several hours. To assure that all nuts have access to adequate pre-soak liquid to cause saturation, the nuts may be occasionally agitated or otherwise mixed so that all nuts are exposed to free liquid. Preferably, the nuts and liquid could be placed in a sealed container which is continually shifted in position, rotated, or agitated to assure that liquid reaches all of the contained nuts.

This invention is useful with other types of nuts such as almonds, but more generally with any form of granular material that must be tested for the presence of bacteria, in situations where the bacteria are most likely to be on the surface of the grains of the granular material and where the interiors of the grains are capable of absorbing liquid, thus secluding enrichment medium into a place where it cannot do any good. The granular material may also be in very fine form, such as in wheat flour or other forms of powders since, on a smaller scale, these are still granular, and have particles of sufficient size to seclude enrichment medium in their interiors.

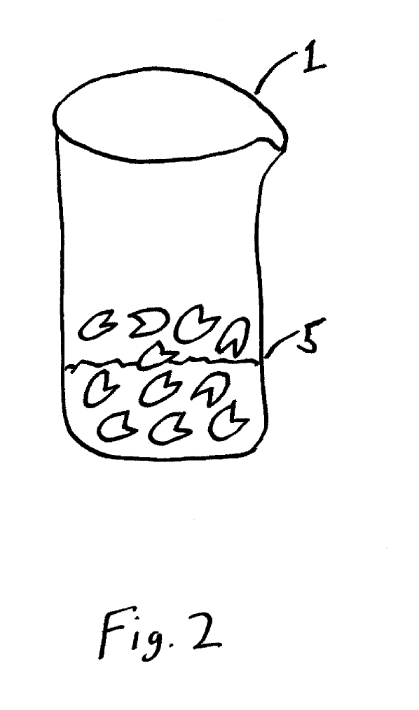

In FIG. 2, beaker 1 is shown after the nuts have had time to absorb the pre-soak liquid. Now, liquid level line 5 is located toward the bottom of the beaker. If no liquid remains present, additional pre-soak liquid can be added and given time to absorb in order to assure saturation.

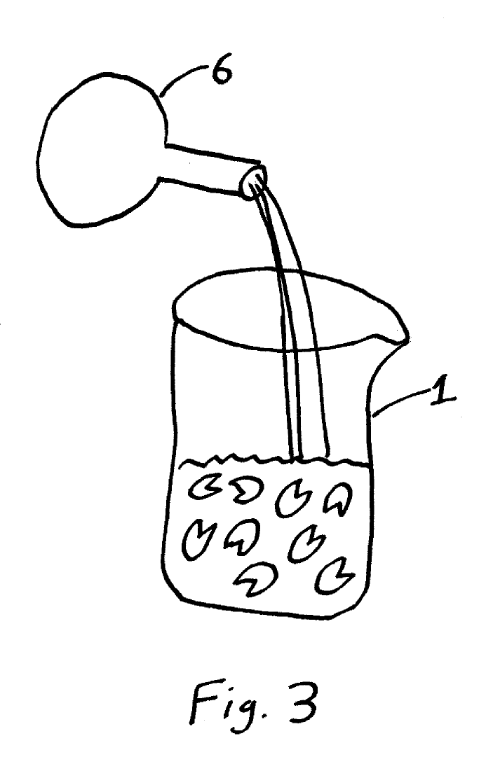

In FIG. 3 vial 6 containing enrichment medium is poured into beaker 1 to provide a suitable environment for the bacteria. The beaker and nuts are then placed in an incubation chamber to await testing. Alternatively, use can be made of enrichment medium material that is in solid form by adding additional water at this point, and then adding solid enrichment material to form the liquid enrichment medium directly with the container with the sample.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact methods illustrated and described, and accordingly, all suitable modifications and equivalents may be resorted to, falling within the scope of the invention. This invention is not limited by the particular physical form of the powder, grain, or other material to be tested. For example, this invention is also applicable to fibers that are capable of absorbing water. A key concept of this invention is the pre-wetting of the material so that water is absorbed into the interior volume of the product rather than more expensive enrichment solution.

I claim:

1. A method for economizing upon the use of enrichment medium when performing microbiological tests upon material which is capable of absorbing water comprising the steps of:
a) exposing the material to water solution;
b) allowing water to be absorbed into the material; and
c) thereafter exposing the material to enrichment medium.

2. A method for economizing upon the use of enrichment medium when performing microbiological testing of nuts comprising the steps of:
a) exposing the nuts to a solution comprising water;
b) waiting until water is absorbed into the nuts; and
c) thereafter exposing the nuts to an enrichment medium capable of supporting growth of at least one microorganism.

3. The method of claims 1 and 2 wherein the water solution comprises sodium chloride dissolved in water in an amount effective to prevent osmotic shock to bacteria.
